(19) United States
(12) Patent Application Publication
(10) Pub. No.: US 2026/0263215 A1
(43) Pub. Date: Sep. 10, 2026

(54) DUAL-STENTED HEART VALVE PROSTHESIS WITH OUTER STENT WITH AN ALTERNATING NODE CONFIGURATION

(71) Applicant: Medtronic, Inc., Minneapolis, MN (US)

(72) Inventors: Brenda L. MCINTIRE, Santa Rosa, CA (US); Jamie S. DUNAWAY, Santa Rosa, CA (US)

(21) Appl. No.: 19/473,187

(22) PCT Filed: Apr. 16, 2024
(86) PCT No.: PCT/IB2024/053711

§ 371 Date: Oct. 7, 2025

Related U.S. Application Data
(60) Provisional application No. 63/496,963, filed on Apr. 19, 2023.

(51) Int. Cl. A61F 2/24

(52) U.S. Cl. CPC A61F 2/2418; A61F 2220/0016

(57) ABSTRACT

A heart valve prosthesis includes an inner stent, an outer stent at least partially surrounding the inner frame, and a prosthetic valve operatively coupled to the inner stent. The outer stent includes a plurality of rows of angled outer struts extending around a circumference of the outer stent, and a plurality of rows of outer nodes coupling ends of the angled outer struts to each other and/or coupling adjacent rows of the angled outer struts to each other. A first row of first outer nodes of the plurality of rows of outer nodes includes a plurality of short outer nodes and a plurality of long outer nodes, wherein the short outer nodes have a first longitudinal length and the long outer nodes have a second longitudinal length, wherein the second longitudinal length is greater than the first longitudinal length.

### CROSS-REFERENCE TO RELATED APPLICATION

This application claims the benefit of U.S. Provisional Patent Application Ser. No. 63/496,963, filed Apr. 19, 2023, the entire content of which is incorporated herein by reference.

### FIELD OF THE INVENTION

The present technology is generally related to prosthetic valves, and in particular is directed towards transcatheter heart valve prosthesis including an inner stent and an outer stent.

### BACKGROUND OF THE INVENTION

The human heart is a four chambered, muscular organ that provides blood circulation through the body during a cardiac cycle. Within the heart there are four valves that control blood flow through the heart's chambers: the mitral valve, the tricuspid valve, the aortic valve, and the pulmonary valve. To ensure blood flows in only one direction, atrioventricular valves (the tricuspid and mitral valves) are present between the junction of the atrium and the ventricles, and semi-lunar valves (pulmonary and aortic valves) govern the exits of the ventricles leading to the lungs and the rest of the body. Each of these valves contain native leaflets that open and close in response to changes in blood pressure as the heart contracts and relaxes. When a valve does not open or close properly, either due to defect or damage, diseases such as stenosis and valvular insufficiency or regurgitation can occur, leading to serious physiological consequences.

Diseases associated with heart valves, such as those caused by damage or a defect, can include stenosis and valvular insufficiency or regurgitation. For example, valvular stenosis causes the valve to become narrowed and hardened which can prevent blood flow to a downstream heart chamber from occurring at the proper flow rate and may cause the heart to work harder to pump the blood through the diseased valve. Valvular insufficiency or regurgitation occurs when the valve does not close completely, allowing blood to flow backwards, thereby causing the heart to be less efficient. A diseased or damaged valve, which can be congenital, age-related, drug-induced, or in some instances, caused by infection, can result in an enlarged, thickened heart that loses elasticity and efficiency. Some symptoms of heart valve diseases can include weakness, shortness of breath, dizziness, fainting, palpitations, anemia and edema, and blood clots which can increase the likelihood of stroke or pulmonary embolism. Symptoms can often be severe enough to be debilitating and/or life threatening.

Prosthetic heart valves have been developed for repair and replacement of diseased or damage heart valves. The prosthetic heart valve can be compressed or reduced in diameter and can be deployed at the site of the disease heart valve through catheter-based delivery systems. Once the prosthetic valve is positioned at the treatment site, for instance, within a mitral valve, the prosthetic heart valve can be expanded to hold the prosthetic heart valve in place.

While these valve prostheses offer minimally invasive methods for heart valve repair and/or replacement, challenges remain such as uniform expansion of a heart valve prosthesis while maintaining required performance in vivo. A challenge relates to providing a valve prosthesis with uniform expansion while maintaining a low profile, reducing crimp strains, and improving fatigue performance and the ability of the valve prosthesis to withstand external forces and prevent migration while reducing the total material present within the mitral valve prosthesis.

### BRIEF SUMMARY OF THE INVENTION

In accordance with a first example hereof, a heart valve prosthesis includes a first end and a second end. The heart valve prosthesis is configured to include a radially compressed configuration and a radially expanded configuration. The heart valve prosthesis comprises an inner stent, an outer stent surrounding at least a portion of the inner stent and operatively coupled to the inner stent, the outer stent configured to secure the heart valve prosthesis to native heart tissue. The outer stent includes a plurality of rows of angled outer struts extending around a circumference of the outer stent, and a plurality of rows of outer nodes coupling ends of the angled outer struts to each other and/or coupling adjacent rows of the angled outer struts to each other, wherein a first row of first outer nodes of the plurality of rows of outer nodes includes a plurality of short outer nodes having a first longitudinal length and the long outer nodes have a second longitudinal length, wherein the second longitudinal length is greater than the first longitudinal length, and a prosthetic valve operatively coupled to the inner stent.

In a second example, in the heart valve prosthesis according to any of the previous or subsequent examples herein, the outer stent includes a first row of first angled outer struts at a first end of the outer stent, a second row of second angled outer struts adjacent the first row of first angled outer struts, and a third row of third angled outer struts adjacent the second row of angled second outer struts, wherein the first row of first outer nodes is located between the second row of second angled outer struts and the third row of third angled outer struts.

In a third example, in the heart valve prosthesis according to any of the previous or subsequent examples herein, first ends of the third angled outer struts are coupled to corresponding first outer nodes of the first row of first outer nodes, and wherein second ends of the third angled outer struts are coupled to a second row of second outer nodes.

In a fourth example, in the heart valve prosthesis according to any of the previous or subsequent examples herein, each of the first outer nodes is coupled to the first end of only a single third angled outer strut.

In a fifth example, in the heart valve prosthesis according to any of the previous or subsequent examples herein, each of the second outer nodes is coupled to the second ends of exactly two of the third angled outer struts.

In a sixth example, in the heart valve prosthesis according to any of the previous or subsequent examples herein, the exactly two third angled outer struts extending between exactly two first outer nodes and a single second outer nodes includes a short third angled outer strut and a long third angled outer strut, wherein the long third angled outer strut is longer than the short third angled outer strut.

In a seventh example, in the heart valve prosthesis according to any of the previous or subsequent examples herein, the third angled outer struts alternate between short third angled outer struts and long third angled outer struts around the circumference of the outer stent, wherein each of the long third angled outer struts is longer than each of the short third angled outer struts.

In an eighth example, in the heart valve prosthesis according to any of the previous or subsequent examples herein, the first ends of the short third angled outer struts are coupled to the long first outer nodes and the first ends of the long third angled outer struts are coupled to the short first outer nodes.

In a ninth example, in the heart valve prosthesis according to any of the previous or subsequent examples herein, each short third outer strut has a longitudinal length of about 4 mm.

In a tenth example, in the heart valve prosthesis according to any of the previous or subsequent examples herein, each long third outer strut has a longitudinal length of about 5 mm.

In an eleventh example, in the heart valve prosthesis according to any of the previous or subsequent examples herein, the first row of first outer nodes alternate between one or more short outer nodes and one or more long outer nodes around the circumference of the outer stent.

In a twelfth example, in the heart valve prosthesis according to any of the previous or subsequent examples herein, the short outer nodes each have a longitudinal length of about 0.5-1.0 mm.

In a thirteenth example, in the heart valve prosthesis according to any of the previous or subsequent examples herein, the long outer nodes each have a longitudinal length of about 1.5-2.0 mm.

In a fourteenth example, in the heart valve prosthesis according to any of the previous or subsequent examples herein, further comprising a plurality of outer node cleats coupled to the outer nodes, wherein in the radially expanded configuration, the outer node cleats extend radially outwardly and towards the first end.

In a fifteenth example, in the heart valve prosthesis according to any of the previous or subsequent examples herein, further comprising a plurality of second outer node cleats coupled to the second outer nodes, wherein in the radially expanded configuration, the outer node cleats extend radially outwardly and towards the first end.

In a sixteenth example, in the heart valve prosthesis according to any of the previous or subsequent examples herein, each of the second outer node cleats extends about 20-25° laterally from a central longitudinal axis of the prosthesis.

In a seventeenth example, in the heart valve prosthesis according to any of the previous or subsequent examples herein, the second outer node cleats alternate between extending in a first lateral direction and a second lateral direction around the circumference of the outer stent.

In an eighteenth example, in the heart valve prosthesis of any one of the previous or subsequent examples herein, the outer stent further a third row of third outer nodes, wherein the third row of third outer nodes is disposed between the first row of first angled outer struts and the second row of second angled outer struts.

In a nineteenth example, in the heart valve prosthesis according to any of the previous or subsequent examples herein, the outer stent includes a first row of first outer cells, wherein each first cell of the first row of outer cells is defined by a single one of the third outer nodes, exactly two of the second angled outer struts, exactly two of the first outer nodes, exactly two of the third angled outer struts, and a single one of the second outer nodes.

In a twentieth example, in the heart valve prosthesis according to any of the previous or subsequent examples herein, the first row of first outer cells consists of exactly twenty-four first outer cells.

In a twenty-first example, in the heart valve prosthesis according to any of the previous or subsequent examples herein, the outer stent further includes fourth row of fourth angled outer struts, wherein the second outer nodes are disposed between the third angled outer struts and the fourth angled outer struts.

In a twenty-second example, in the heart valve prosthesis according to any of the previous or subsequent examples herein, each of the second outer nodes is coupled to a first end of a single fourth angled outer strut of the fourth row of fourth angled outer struts.

In a twenty-third example, in the heart valve prosthesis according to any of the previous or subsequent examples herein, the outer stent further includes a fourth row of fourth outer nodes, wherein each fourth outer node is coupled of a second end of a pair of the fourth angled outer struts.

In a twenty-fourth example, in the heart valve prosthesis according to any of the previous or subsequent examples herein, the outer stent further include a second row of second outer cells, wherein each second outer cell is defined by a single one first outer nodes, two third angled outer struts, two second outer nodes, two fourth angled outer struts, and a single one of the fourth outer nodes.

In a twenty-fifth example, in the heart valve prosthesis according to any of the previous examples herein, the second row of second outer cells consists of exactly twelve second outer cells.

### BRIEF DESCRIPTION OF DRAWINGS

The foregoing and other features and advantages of the invention will be apparent from the following description of embodiments thereof as illustrated in the accompanying drawings. The accompanying drawings, which are incorporated herein and form a part of the specification, further serve to explain the principles of the invention and to enable a person skilled in the art to make and use the invention. The drawings are not to scale.

FIG. 1A depicts a side view of a frame of a heart valve prosthesis in accordance with aspects of the disclosure, the frame including an inner frame and an outer frame.

FIG. 1B depicts a side view of the inner frame of FIG. 1A in accordance with aspects of the disclosure.

FIG. 1C depicts a side view of the outer frame of FIG. 1A in accordance with aspects of the disclosure.

FIG. 2 depicts a flat, as-cut view of the outer frame of the heart valve prosthesis in accordance with aspects of the disclosure.

FIG. 3 depicts a close-up view of the outer frame of the heart valve prosthesis in accordance with aspects of the disclosure.

FIG. 4A depicts a perspective view of an outflow end of the outer frame in accordance with aspects of the disclosure.

FIG. 4B depicts a top view of an inflow end of the outer frame of FIG. 4A in accordance with aspects of the disclosure.

### DETAILED DESCRIPTION OF THE INVENTION

Specific embodiments of the present invention are now described with reference to the figures, wherein like reference numbers indicate identical or functionally similar elements. The terms “proximal” and “distal”, when used in the following description to refer to the heart valve prosthesis or elements of the heart valve prosthesis are with reference to the direction of the direction of blood flow. Thus, “proximal” refers to positions in an upstream direction with respect to the blood flow and “distal” refers to positions in a downstream direction with respect to blood flow.

As used in this specification, the singular forms “a”, “an” and “the” specifically also encompass the plural forms of the terms to which they refer, unless the content clearly dictates otherwise. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 5%. It should be understood that use of the term “about” also includes the specifically recited number of value.

The following detailed description is merely exemplary in nature and is not intended to limit the invention or the application and use of the invention. Although the description of the invention is in the example context of the treatment of a mitral valve, the invention may be used in other anatomical sites. For example, the present invention may be applied in other locations located within the body, for example, heart valves other than the mitral valve, which are known as the aortic valve, tricuspid valve, and/or pulmonary valve. Furthermore, there is no intention to be bound by any expressed or implied theory presented in the preceding technical field, background, brief summary, or the following detailed description.

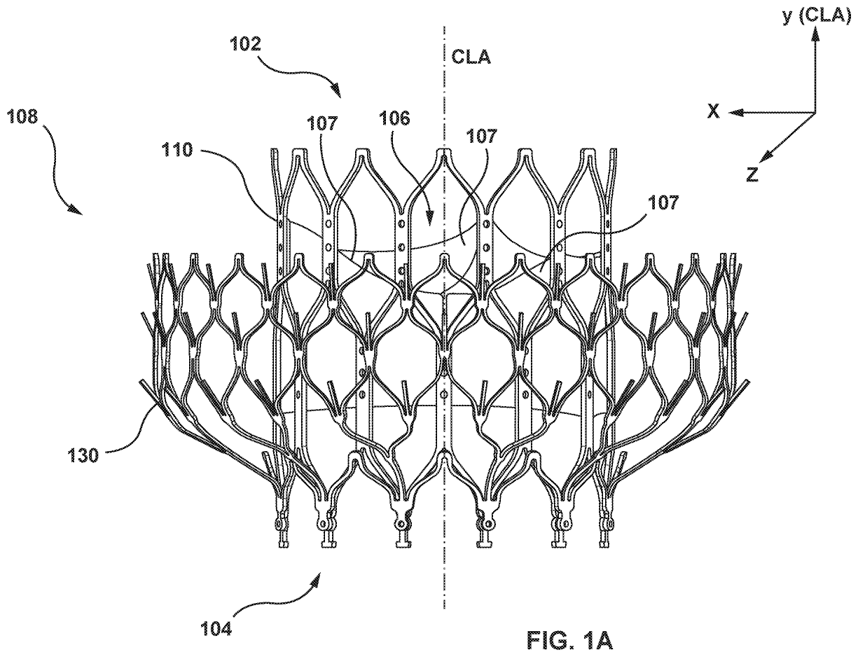

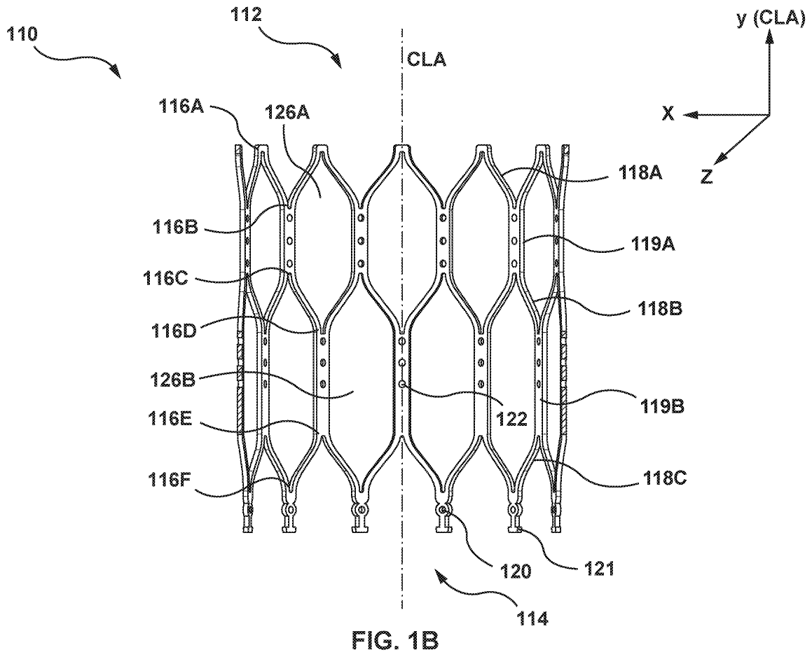

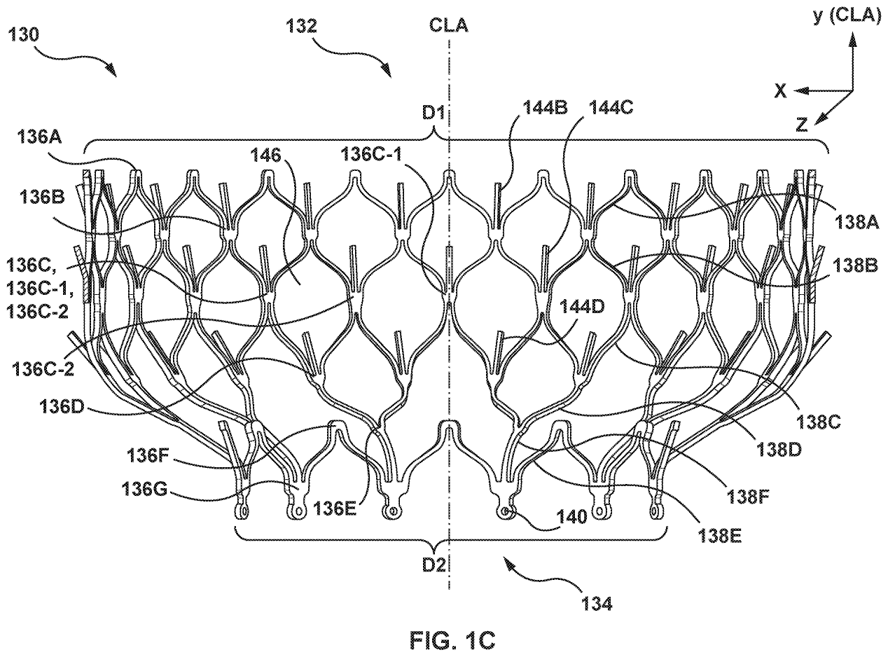

A side view of a transcatheter heart valve prosthesis 100 in accordance with an aspect of the disclosure is shown in FIG. 1A. The heart valve prosthesis 100 is configured to be compressed into a reduced-diameter delivery configuration within a delivery catheter and to return to a radially expanded, deployed configuration when delivered/released from the delivery catheter within a native heart valve. The heart valve prosthesis 100 includes a frame 108 and a prosthetic valve 106. The frame 108 has a stent-like structure that is configured to support the prosthetic valve 106 and to define, along a central longitudinal axis CLA thereof, a blood flow lumen that substantially extends from an inflow end 102 to an outflow end 104 of the heart valve prosthesis 100. In the embodiments shown herein, the frame 108 generally includes an inner stent 110 and an outer stent 130. In aspects hereof, the inner stent 110 of the frame 108 may alternatively be referred to as a valve support, an inner frame, and/or a valve housing. Similarly, the outer stent 130 of the frame 108 may alternatively be referred to as an anchoring frame, an anchoring member, a fixation ring, and/or an outer frame. The inner stent 110 is configured to hold the prosthetic valve 106, and the outer stent 130, which surrounds the inner stent 110, is configured to secure the heart valve prosthesis 100 to the native tissue of the heart when implanted in vivo. The frame 108 may be considered to have a dual-stent structure, i.e., an inner stent and an outer stent.

FIG. 1B shows a side view of the inner stent 110 only in accordance with an aspect of the disclosure. The inner stent 110 is positioned within the outer stent 130 such that at least a portion thereof is spaced from the outer stent 130. The inner stent 110 generally forms a hollow cylindrical shape having a substantially constant diameter from an inflow end 112 to an outflow end 114 thereof. The stent-like structure of the inner stent 110 defines a plurality of open cells 126 arranged in a repeating pattern around a circumference of the inner stent 110. In the embodiment shown, the inner stent 110 includes twenty-four (24) cells 126 around a circumference of shown, the inner stent 110 (only half are shown in the side view of FIG. 1B). More particularly, the inner stent 110 includes exactly twelve (12) proximal cells 126A and exactly twelve (12) distal cells 126B, which will be described in further detail below.

Starting at the inflow end 112, the inner stent 110 includes a first or proximal row of first or proximal angled inner struts 118A connected to each other at their proximal ends at first inner nodes 116A and at their distal ends at second inner nodes 116B. A second row of second angled inner struts 118B are connected to each other at their proximal ends at third inner nodes 116C and at their distal ends at fourth inner nodes 116D. A third row of third angled inner struts 118C are connected to each other at their proximal ends at fifth inner nodes 116E and at their distal ends at sixth inner nodes 116F. Further, first connectors 119A couple respective second inner nodes 116D to respective third inner nodes 116C, and second connectors 119B couple respective fourth inner nodes 116D to respective fifth inner nodes 116E The first and second inner connectors 119A, 119B are generally parallel to the central longitudinal axis CLA. The first and second inner connectors 119A, 119B may also act as commissure posts for attaching the prosthetic valve 106 to the inner stent 110. Thus, the first and second inner connectors 119A, 119B may include suture holes or apertures 122 to aid in attaching the prosthetic valve 106 to the inner stent 110. Although all of the first and second inner connectors 119A, 119B of the inner stent 110 are shown with apertures 122, in other embodiments, only the first and second inner connectors 119A, 119B that are used as commissure posts have the apertures 122. The pattern and relative spacing of apertures may also vary compared to what is shown in, e.g., FIG. 1B. Still other embodiments include commissure windows, or slots, located at the first and/or second connectors, one 119A and/or 119B, respectively. Apertures and/or commissures, as the case may be, can be used to attach sutures for securing one or more commissures of a valve for controlling blood flow through the inner stent and/or material, such as an anti-paravalvular leakage component made of polyethylene terephthalate (PET) or other fabric or plastic, for example.

Extending distally from each of the sixth inner nodes 116F are eyelets 120. The eyelets 120 may be used to couple the inner stent 110 to the outer stent 130 by aligning them with corresponding eyelets on the outer stent 130, as shown in FIG. 1A, and then attaching them using hooks, loops, sutures, or rivets, for example. In particular, the eyelets 120 of the inner stent 110 may be aligned with eyelets 140 of the outer stent 130, which are described in further detail below, and are coupled to each other, for example, using a rivet. Extending distally from the eyelets 120 are T-bar connectors 121. The T-bar connectors 121 may be used to connect the transcatheter heart valve prosthesis 100 to a delivery system for delivering the transcatheter heart valve prosthesis 100 into a patient's body. In the embodiment shown, each of the eyelets 120 includes a T-bar connector 121 extending therefrom. Thus, there are twelve (12) T-bar connectors 121. However, this is not meant to be limiting, and there may be more or fewer T-bar connectors 121. The T-bar connectors 121 in the embodiment shown are T-shaped, but such connectors may be shapes other than T-shaped, such as L-shaped, O-shaped, V-shaped or otherwise shaped. The description of the struts, cells and nodes of the inner stent 110 above is merely an example, and is not meant to be limiting. Other arrangements may also be utilized.

As shown in FIG. 1B, the inner stent 110 includes a row of twelve (12) proximal cells 126A disposed at the inflow or proximal end 112 of the inner stent 110, and a row of twelve (12) distal cells 126B disposed at the outflow or distal end 114 of the inner stent 110. Each proximal cell 126A of the inner stent 110 is defined by one first inner node 116A, a pair of adjacent first angled inner struts 118A joined by the first inner node 116A, a pair of adjacent second inner nodes 116B at the distal end of the pair of adjacent first angled inner struts 118A, a pair of first connectors 119A, a pair of adjacent third inner nodes 116C at the distal ends of the first connectors 119A, a pair of adjacent second angled inner struts 118C, and one fourth inner node 116D that joins the pair of adjacent third angled inner struts 118C. Each distal cell 126B of the inner stent 110 is defined by one third inner node 116C, a pair of adjacent second angled inner struts 118B joined by the third inner node 116C, a pair of adjacent fourth inner nodes 116D at the distal end of the second angled inner struts 118B, a pair of adjacent second connectors 119B extending from the fourth inner nods 116D, a pair of adjacent fifth inner nodes 116E at the distal ends of the pair of adjacent second connectors 119B, a pair of adjacent third angled inner struts 118C, and one sixth inner node 116F that joins the adjacent third angled inner struts 118C. As can be seen, in the embodiment shown, the second inner connectors 119B have a length that is longer than a length of the first inner connectors 119A. Thus, the area of each of the distal cells 126B is larger than the area of each of the proximal cells 126A. In an embodiment, the first angled inner struts 118A, second angled inner struts 118B, and third angled inner struts 118C each have a length of about 5.2 mm. In an embodiment, the first inner connectors 119A each have a length of about 5.7 mm plus or minus 0.05 mm and the second inner connectors 119B each have a length of about 8.9 mm plus or minus 0.05 mm. In an embodiment, the inner stent 110 has a total length, from the inflow end 112 to the outflow end 114, of about 31.3 mm plus or minus 0.25 mm and a diameter of about 28.0 mm plus or minus 0.3 mm in the radially expanded configuration.

In the embodiment shown, the inner stent 110 includes twelve (12) first inner nodes 116A, with each first inner node 116A joining two (2) adjacent first angled inner struts 118A such that the inner stent 110 includes twenty-four (24) first angled inner struts 118A. Further, the inner stent 110 includes twelve (12) sixth inner nodes 116F, with each sixth inner node 116F joining two (2) adjacent third angled inner struts 118C such that the inner stent 110 includes twenty-four (24) third angled inner struts 118C. Further, the inner stent 110 includes twelve (12) first inner connectors 119A, each second inner connector 118B joining a pair of adjacent first angled inner struts 118A and a pair of adjacent second angled inner struts 118B such that the inner stent 110 includes twenty-four (24) second angled inner struts 118B. Further, the inner stent 110 includes twelve (12) second inner connectors 119B, each second inner connector 119A joining a pair of adjacent second angled inner struts 118B and a pair of adjacent third angled inner struts 118C. Thus, the inner stent 110 includes twelve (12) first inner nodes 116A, twelve (12) second inner nodes 116B, twelve (12) third inner nodes 116C, twelve (12) fourth inner nodes 116D, twelve (12) fifth inner nodes 116E, and twelve (12) sixth inner nodes 116F. In other embodiments, the numbers of nodes and struts vary from this example.

In accordance with aspects hereof, the outer stent 130 and the inner stent 110 of the frame 108 of the heart valve prosthesis 100 may be made from any number of suitable biocompatible materials, e.g., stainless steel, nickel titanium alloys such as Nitinol™, cobalt chromium alloys such as MP35N, other alloys such as ELGILOY® (Elgin, Ill.), various polymers, pyrolytic carbon, silicone, polytetrafluoroethylene (PTFE), or any number of other materials or combination of materials. Suitable biocompatible materials for the outer stent 130 and the inner stent 110 would be selected to enable the heart valve prosthesis 100 to be compressed into a reduced diameter configuration for transcatheter delivery to a native valve, whereby release from a delivery catheter allows the outer stent 130 and the inner stent 110, and hence the heart valve prosthesis 100, to self-expand, returning to an expanded, deployed configuration. In some embodiments, the self-expansion is accomplished through the use of a shape-memory material such as Nitinol™. The inner stent 110 and the outer stent 130 of the heart valve prosthesis 100 may be processed to have a default or “set” shape that coincides with the radially expanded, deployed configuration. Therefore, once the compressed heart valve prosthesis 100 is delivered and released, the prosthesis 100 will return to the default or “set” deployed configuration. The outer stent 130 and the inner stent 110 may be the same material or may be different materials.

As introduced above, the prosthetic valve 106 of the heart valve prosthesis 100 is capable of regulating flow through the inner stent 110 via valve leaflets 107. FIG. 1A illustrates an exemplary prosthetic valve 106 having three leaflets 107, although a bicuspid leaflet configuration may alternatively be used in embodiments hereof. When deployed in situ, the prosthetic valve 106 in a closed state is configured to block blood flow in one direction to regulate blood flow through a blood flow lumen of the inner stent 110. The valve leaflets 107 are disposed to coapt within the inner stent 110 and are secured to the inner surface of the inner stent 110, such that the valve leaflets 107 open during a desired phase of the cardiac cycle, depending on the native valve being replaced. Adjoining pairs of leaflets 107 may be attached to one another at their lateral ends to form leaflet commissures. The leaflet commissures in turn may be attached to the inner stent 110 at the connectors 118B, 118D. The orientation of the leaflets 107 within the inner stent 110 depends upon which end of the heart valve prosthesis 100 is the inflow end 102 and which end of the heart valve prosthesis 100 is the outflow end 104, thereby ensuring one-way flow of blood through the heart valve prosthesis 100.

The leaflets 107 may be formed of various flexible materials including, but not limited to, natural pericardial material such as tissue from bovine, equine or porcine origins, or synthetic materials such as polytetrafluoroethylene (PTFE), DACRON® polyester, pyrolytic carbon, or other biocompatible materials. With certain prosthetic leaflet materials, it may be desirable to coat one or both sides of the replacement valve leaflet with a material that will prevent or minimize overgrowth. It is further desirable that the prosthetic leaflet material is durable and not subject failure due to stretching, deforming, or fatigue.

In some embodiments, the heart valve prosthesis 100 may further include a skirt (not shown) that is coupled to the inner stent 110. The skirt acts as a seal around the heart valve prosthesis 100 to limit potential paravalvular leaks. The skirt may be a natural or biological material such as pericardium or another membranous tissue such as intestinal submucosa. Alternatively, the skirt may be a low-porosity woven fabric, such as polyester, PET, Dacron fabric or PTFE, which creates a one-way fluid passage when attached to the prosthetic heart valve 100. In one embodiment, the skirt may be a knit or woven polyester, such as a polyester or PTFE knit, which can be utilized when it is desired to provide a medium for tissue ingrowth and the ability for the fabric to stretch to conform to a curved surface. Polyester velour fabrics may alternatively be used, such as when it is desired to provide a medium for tissue ingrowth on one side and a smooth surface on the other side.

FIG. 1C depicts the outer stent 130 of the frame 108 according to embodiments hereof. The outer stent 130 is configured to secure the heart valve prosthesis 100 to the native valve and the surrounding tissue, such as the inward facing-surface of the native leaflets and or the native valve annulus. The outer stent 130 is positioned around the inner stent 110 and defines an inflow or proximal end 132 having a first diameter DI and an outflow or distal end 134 having a second diameter D2 that is smaller than the first diameter D1. The diameter of the outer stent 130 may vary from the inflow end 132 to the outflow end 134 of the outer stent 130 based on how the outer stent 130 is shape-set on a mandrel. In other embodiments, the outer stent may have, as examples, a circular, oval, D-shape, convex, or concave circumference, and it may be right-cylindrical, hourglass-shaped, convex, or concave, as examples, in its side profile.

At least a portion of an outer surface of the outer stent 130, when the heart valve prosthesis 100 is in an expanded state, is configured to be disposed against the native tissue of the heart for securing the outer stent 130 and, concurrently, the heart valve prosthesis 100, at the native heart valve. Further, a portion of the outer stent 130, in this embodiment the inflow portion, is mechanically decoupled from the inner stent 110, as shown in FIG. 1A. In particular, the outer stent 130 may deform upon implantation within a native mitral valve annulus, and/or expand and contract in response to movement of the native tissue, while remaining spaced from, and not touching, the inner stent 110, thereby enabling the inner stent 110 to remain a relatively undeformed framework for attaching a prosthetic valve. This helps ensure that the attached prosthetic valve coapts as anticipated in its closed configuration. Therefore, the inner stent 110 is mechanically uncoupled from external forces, in particular, external forces are fully or partly absorbed by the outer stent 130 and at the connection between the outer stent 130 and the inner stent 110 to more efficiently replicate the function of the native heart valve. In addition, the outer stent 130 may further include a plurality of barbs, prongs, cleats, or, more generally, protrusions, that extend radially from the outer surface of the outer stent 130 and are configured to engage with the native tissue, further fixating and/or arresting movement of the outer stent 130 with respect to the surrounding native tissue, as explained in further detail below.

As shown in FIG. 1C, the outer stent 130 defines a plurality of open cells 146 arranged in a repeating pattern around a circumference of the outer stent 130. In the embodiment shown, the outer stent 130 includes three rows of cells 146, which will be described below. Each cell 146 of the outer stent 130 is defined by a plurality of struts and nodes connecting adjacent struts. Starting at the inflow end 132, the outer stent 130 includes a first row of first angled outer struts 138A, wherein adjacent proximal ends of the first angled outer struts 138A are connected to each other at first outer nodes 136A and adjacent distal ends of the first angled struts 138A are connected to each other at second outer nodes 136B. The outer stent 130 includes a second row of second angled outer struts 138B, wherein adjacent proximal ends are of the second angled struts connected to each other and to the distal ends of the first angled outer struts 138A at the second outer nodes 136B. A third row of third angled outer struts 138C are connected to each other at their proximal ends and to the distal ends of the second outer struts 138B at third outer nodes 136C. Adjacent distal ends of the third angled struts 138C are connected to each other at fourth outer nodes 136D. Extending distally from each of the fourth outer nodes 136D is a single fourth angled outer strut 138D of a fourth row of fourth angled outer struts 138D. Adjacent distal ends of the fourth angled outer struts 138B are connected to each other and to one outer connector arm 138F of a plurality of outer connector arms 138F at fifth outer nodes 136E. Each outer connector arm 138F extends distally and substantially longitudinally or parallel to the central longitudinal axis CLA of the outer frame 130 from a respective fifth outer node 136E. A distal end of each connector arm 138F is coupled to distal ends of adjacent pairs of fifth angled outer struts 138E at seventh outer nodes 136G disposed at the outflow end 134 of the outer stent 130. Each pair of adjacent fifth outer struts 138E are connected to each other at their proximal ends at sixth outer nodes 136F, as shown in FIG. 1C. The sixth outer nodes 136F are not attached to struts proximal of the sixth outer nodes 334F. The outer frame 130 further includes a plurality of cleats 144, which will be described in further detail below. Like cleats 144, the sixth outer nodes 136F may extend outward from the central longitudinal axis and thereby provide a fixation or anchoring function at the portion of the outer stent 130 from which they extend.

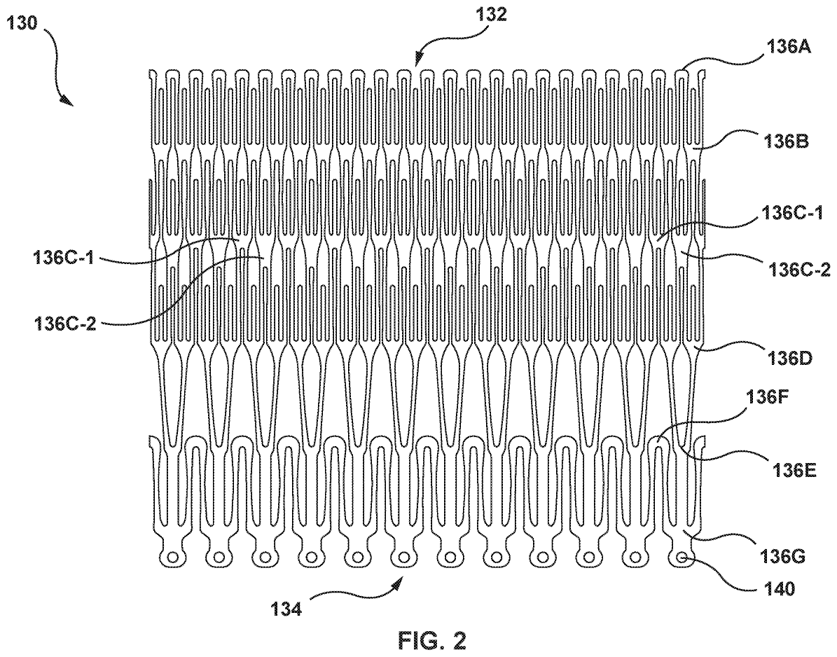

FIG. 2 shows an as-cut, flat view of the outer frame 130. As can be seen, the inflow end 132 of the outer frame 130 includes exactly twenty-four (24) first outer nodes 136A and the outflow end 134 of the outer frame 130 includes exactly twelve (12) seventh outer nodes 136G. Extending distally from the seventh outer nodes 136G are eyelets 140. The eyelets 140 of the outer frame 130 are configured for coupling the outer frame 130 to the inner frame 110. In particular, the eyelets 140 of the outer frame 130 may be aligned with the eyelets 120 of the inner stent 110 and may be coupled to each other, for example, by using a rivet. The description of the struts, cells and nodes of the outer frame 130 above is merely an example, and is not meant to be limiting. Other arrangements may also be utilized.

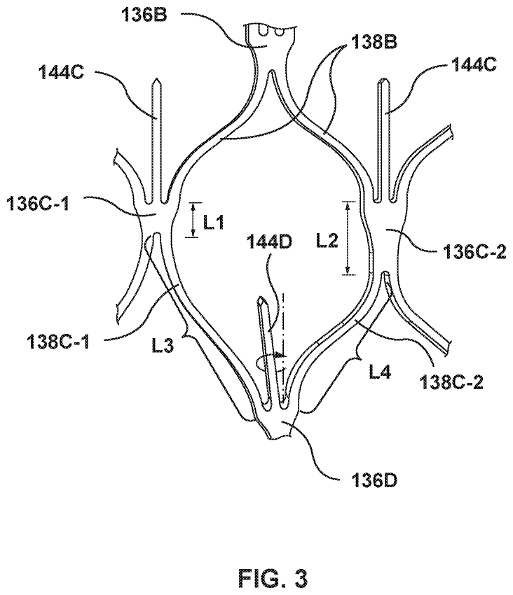

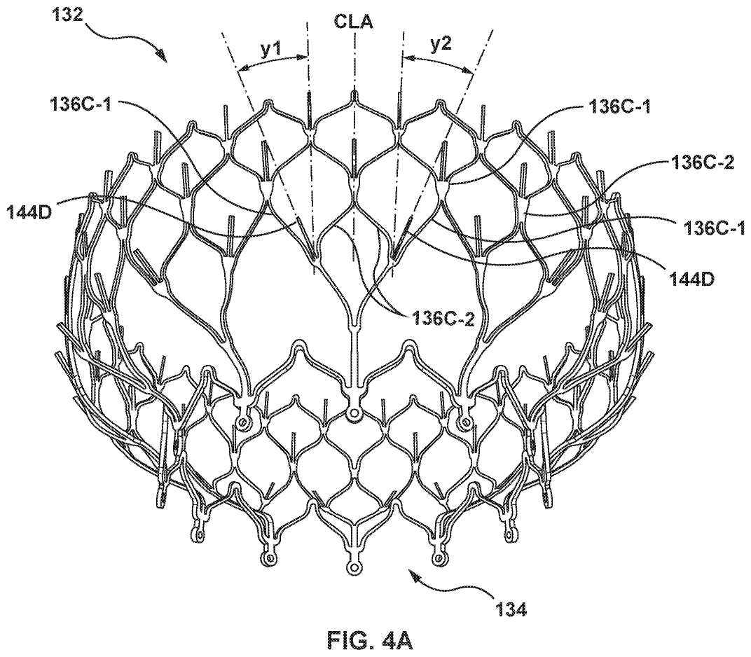

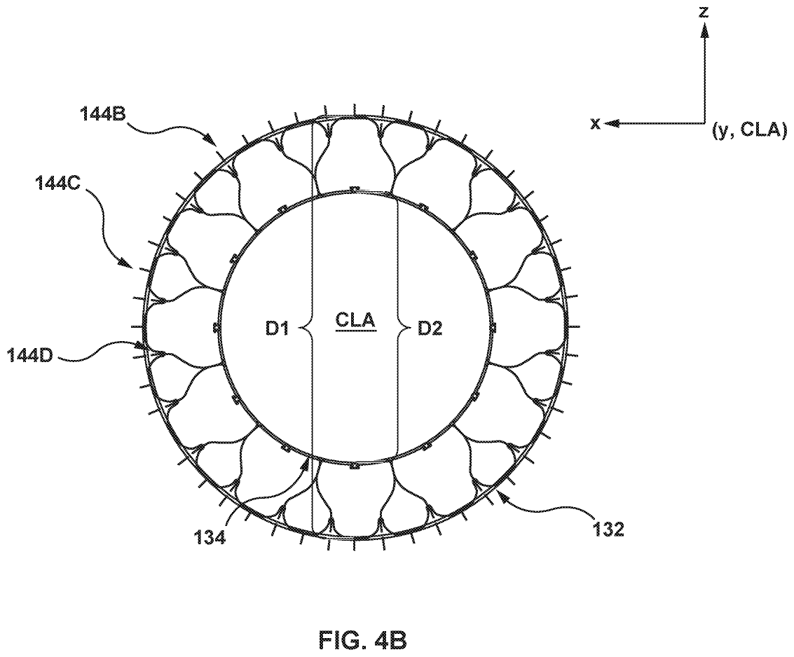

FIG. 3 shows a close-up view of two adjacent third outer nodes 136C of the outer frame 130 with the outer frame 130 in the radially expanded configuration according to an embodiment hereof. As can be seen, the entire row of third outer nodes 136C of the outer frame 130 alternate at a 1:1 ratio between a short third outer node 136C-1 and a long third outer node 136C-2. In other words, every other third outer node 136C of the outer frame 130 is a short third outer node 136C-1 and every third outer node 136C between each pair of short third outer node 136C-1 is a long third outer node 136C-2. Each short third outer node 136C-1 is longitudinally aligned with each sixth outer node 136F and each long third outer node 136C-2 is longitudinally aligned with each fifth outer node 136E, as best shown in FIG. 4B. Stated another way, each short third outer node 136C-1 is disposed proximal to each sixth outer node 136F of the outer frame 130 and each long third outer node 136C-2 is disposed proximal to each fifth outer node 136E of the outer frame 130. In embodiments, each short third outer node 136C-1 has a first longitudinal length L1 of about 0.5-1.0 mm and each long third outer node 136C-2 has a second longitudinal length L2 of about 1.5-2.0 mm, as shown in FIG. 3. In an embodiment, the first longitudinal length LI is about 0.74 mm and the second longitudinal length L2 is about 1.74 mm. Although a 1:1 alternation between short and long notes is depicted here, other alternation ratios, such as 1:12, 1:6, 1:2, 1:3, and 2:3, as examples, are contemplated.

As shown in FIG. 3, each adjacent pair of short third outer nodes 136C-1 and long third outer nodes 136C-2 is connected to a fourth outer node 136D by two adjacent third outer struts 138C. More particularly, a long third outer strut 138C-1 connects the short third outer node 136C-1 to the fourth outer node 136D of the outer frame 130 and a short third outer strut 138C-2 connects the long third outer node 136C-2 to the fourth outer node 136D of the outer frame 130, as shown and described with respect to FIG. 3. Due to the shorter length of the short third outer node 136C-1, the long third outer strut 136C-1 has a longitudinal length that is greater than a longitudinal length of the short third outer strut 136C-2. The long third outer strut 136C-1 may have a longitudinal length L3 of about 5 mm and the short third outer strut 136C-2 may have a longitudinal length L4 of about 4 mm, as best shown in FIG. 3. By alternating each third outer node 136C of the outer frame 130 between a short third outer node 136C-1 and a long third outer node 136C-2, the angulation of the cleats 144 of the outer frame 130 of the outer stent 130 are more uniform compared to cleat angles on an outer stent that lacks alternating node lengths, thus reducing migration risk. Further, the cells 146 between the third outer nodes 136C and the fourth outer nodes 136D expand more uniformly when the outer frame 130 transitions from a radially compressed configuration to a radially expanded configuration. This alternating stent node design allows for more uniform cell expansion, reduces crimp strains, and improves fatigue performance and migration resistance.

As stated previously, the outer stent 130 includes a plurality of cleats 144 that extend radially outwards and proximally from the outer surface of the outer frame 130 and are configured to engage with the native tissue. As shown best in FIG. 1C, the outer frame 130 includes a row of second outer node cleats 144B, a row of third outer node cleats 144C, and a row of fourth outer node cleats 144D. Each cleat 144 is a substantially straight segment that extends substantially proximally (i.e., towards the inflow end 132 of the outer frame 130) with respect to the central longitudinal axis CLA (y-axis) and radially outwardly (i.e., in the z-axis direction). In other words, each cleat of the plurality of cleats 144 does not extend in a direction towards the outflow end 134 of the outer frame 130, nor do they extend inwardly towards the central longitudinal axis CLA of the frame 130. As shown, in the radially expanded configuration, each second outer node cleat 144B is coupled to a corresponding second outer node 136B and extends proximally with respect to the y-axis and outwardly with respect to the z-axis, i.e., radially outwardly. Similarly, in the radially expanded configuration, each third outer node cleat 144C is coupled to a corresponding third outer node 136C and extends proximally with respect to the y-axis and outwardly with respect to the z-axis, i.e., radially outwardly. Similarly, in the radially expanded configuration, each fourth outer node cleat 144D is coupled to a corresponding fourth outer node 136D and extends proximally with respect to the y-axis and outwardly with respect to the z-axis, i.e., radially outwardly. Thus, each of the cleats 144 extends proximally and radially outwardly.

With respect to the x-axis (i.e., lateral plane), each second outer node cleat 144B and each third outer node cleat 144C extends substantially parallel to the CLA of the frame 130. In other words, each second outer node cleat 144B and each third outer node cleat 144C does not extend substantially laterally or substantially circumferentially. However, those skilled in the art will recognize that stents do not necessarily expand uniformly. Therefore, in the radially expanded or deployed configuration, the second and third outer node cleats 144B, 144C may be angled laterally or circumferentially (i.e., in the x-axis direction) with respect to the central longitudinal axis CLA (i.e., with respect to the y-axis). In some embodiments, that angle is about or below 3 degrees.

As best shown in FIGS. 3 and 4A, each fourth outer node cleat 144D is coupled to each fourth outer node 136D of the outer frame 130 and extends proximally between one long third outer strut 138C-1 and one short third outer strut 138C-2 of the outer frame 130. As stated previously, the long third outer strut 138C-1 couples the fourth outer node 136D to the short third outer node 136C-1 and the short third outer strut 138C-2 couples the fourth outer node 136D to the long third outer node 136C-2. When the outer frame 130 radially expands from the radially compressed configuration to the radially expanded or deployed configuration, the short third outer strut 138C-2 curves more than the long third outer strut 138C-1 with respect to the central longitudinal axis, as shown in FIG. 3. This curvature angles the fourth outer node cleat 144D towards the long third outer strut 138C-1, as shown. However, the disproportionate lengths of the long third outer strut 138C-1 and the short third outer strut 138C-2 reduces the angle that the fourth outer node cleat 144D extends laterally or circumferentially (i.e., in the x-axis direction). In other words, the asymmetric strut lengths of the long and short third outer struts 138C-1, 138C-2 yields an increased stiffness in the short third outer strut 138C-2, causing the fourth outer node cleat 144D to naturally straighten with respect to the x-axis of the outer frame 130 during expansion of the outer frame 130. As a result, each fourth outer node cleat 144D extends at an angle γ1, γ2 of about 8°-12° laterally (i.e., in the x-axis direction) from the central longitudinal axis CLA, as shown in FIG. 4A. The angle γ1, γ2 at which each fourth outer node cleat 144D extends laterally in the described configuration is reduced compared to an outer frame that does not include a short node, a long node, a short strut, and a long strut configuration. Reducing the angle that each fourth outer node cleat 144D extends in the circumferential direction improves the fourth outer node cleats' 144D ability to engage with the native anatomy, therefore improving migration resistance of the prosthesis 100. Accordingly, as shown in FIG. 4A, every other fourth outer node cleat 144D extends at an angle γ1 to the left (when the outer frame 130 is viewed from the side) towards the long third outer strut 138C-1 it is adjacent to, and every other fourth outer node cleat 144D extends at an angle γ2 to the right (when the outer stent 130 is viewed from the side) towards the long third outer strut 138C-1 it is adjacent to.

FIG. 4B shows the outer frame 130 as viewed from the inflow end 132 of the frame 130. As can be seen, an outer diameter DI of the outflow end 134 of the outer frame 130 is smaller than an outer diameter D2 of the inflow end 132 of the outer frame 130. The outer diameter D1 of the outflow end 134 of the outer frame 130 can range from about 27.5 mm 28.5 mm and the outer diameter D2 of the inflow end 132 of the outer frame 130 can range from about 43.5 mm- 44.5 mm. Additionally, it can be seen that the second, third and fourth outer node cleats 144B, 144C, 144D all extend radially outward (i.e., in the z-direction) from the outer frame 130 with respect to the central longitudinal axis CLA of the frame 130.

The second outer node cleats 144B may have a length of about 2.8 mm the third outer node cleats 144C may have a length of about 2.8 mm, and the fourth outer node cleats 144D may have a length of about 2.9 mm. The fourth outer node cleats 144D may have a forked configuration, wherein the fourth outer node cleats 144D extend and branch into a “Y-shaped” configuration, to aid in engaging the native tissue of the target site. According to the embodiment shown and described herein, the outer frame 130 includes exactly twenty-four (24) second outer node cleats 144B, exactly twenty-four (24) third outer node cleats 144C, and exactly twenty-four (24) fourth outer node cleats 144D, as best shown in FIG. 2. The plurality of cleats 144 are shape set to the radially expanded configuration. A tool may be used to move the cleats 144 to the radially expanded configuration prior to shape setting the outer frame 130 to the radially expanded configuration. Those skilled in the art will recognize that the plurality of cleats 144 may be varied from the embodiment described. The embodiment shown and described herein is not meant to be limiting, and other embodiments may be utilized in keeping with the scope of the present disclosure.

While various embodiments according to the present invention have been described above, it should be understood that they have been presented by way of illustration and example only, and not limitation. It will be apparent to person skilled in the relevant art that various changes in form and detail can be made therein without departing from the spirit and scope of the invention. Thus, the breadth and scope of the present invention should not be limited by any one of each embodiment discussed herein, and of each reference cited herein, can be used in combination with the features of any other embodiment. All patents and publication discussed herein are incorporated by reference herein in their entirety.

1. A heart valve prosthesis comprising a first end and a second end, the heart valve prosthesis configured to include a radially compressed configuration and a radially expanded configuration, the heart valve prosthesis comprising:
an inner stent;
an outer stent surrounding a least a portion of the inner stent and operatively coupled to the inner stent, the outer stent configured to secure the heart valve prosthesis to native heart tissue, the outer stent including:
a plurality of rows of angled outer struts extending around a circumference of the outer stent; and
a plurality of rows of outer nodes coupling ends of the angled outer struts to each other and/or coupling adjacent rows of the angled outer struts to each other, wherein a first row of first outer nodes of the plurality of rows of outer nodes includes a plurality of short outer nodes and a plurality of long outer nodes, wherein the short outer nodes have a first longitudinal length and the long outer nodes have a second longitudinal length, wherein the second longitudinal length is greater than the first longitudinal length; and
a prosthetic valve operatively coupled to the inner stent.

2. The heart valve prosthesis of claim 1, wherein the outer stent includes a first row of first angled outer struts at a first end of the outer stent, a second row of second angled outer struts adjacent the first row of first angled outer struts, and a third row of third angled outer struts adjacent the second row of angled second outer struts, wherein the first row of first outer nodes is located between the second row of second angled outer struts and the third row of third angled outer struts.

3. The heart valve prosthesis of claim 2, wherein first ends of the third angled outer struts are coupled to corresponding first outer nodes of the first row of first outer nodes, and wherein second ends of the third angled outer struts are coupled to a second row of second outer nodes.

4. The heart valve prosthesis of claim 3, wherein each of the first outer nodes is coupled to the first end of exactly two third angled outer struts.

5. The heart valve prosthesis of claim wherein each of the second outer nodes is coupled to the second ends of exactly two of the third angled outer struts.

6. The heart valve prosthesis of claim 5, wherein the exactly two third angled outer struts extending between exactly two first outer nodes and a single second outer nodes includes a short third angled outer strut and a long third angled outer strut, wherein the long third angled outer strut is longer than the short third angled outer strut.

7. The heart valve prosthesis of claim 2, wherein the third angled outer struts alternate between short third angled outer struts and long third angled outer struts around the circumference of the outer stent, wherein each of the long third angled outer struts is longer than each of the short third angled outer struts.

8. The heart valve prosthesis of claim 3, wherein the first ends of the short third angled outer struts are coupled to the long first outer nodes and the first ends of the long third angled outer struts are coupled to the short first outer nodes.

9. (canceled)

10. (canceled)

11. The heart valve prosthesis of claim 1, wherein the first row of first outer nodes alternate between one or more short outer nodes and one or more long outer nodes around the circumference of the outer stent.

12. (canceled)

13. (canceled)

14. The heart valve prosthesis of claim 1, further comprising a plurality of outer node cleats coupled to the outer nodes, wherein in the radially expanded configuration, the outer node cleats extend radially outwardly and towards the first end.

15. The heart valve prosthesis of claim 14, further comprising a plurality of second outer node cleats coupled to the second outer nodes, wherein in the radially expanded configuration, the outer node cleats extend radially outwardly and towards the first end.

16. (canceled)

17. The heart valve prosthesis of claim 17, wherein the second outer node cleats alternate between extending in a first lateral direction and a second lateral direction around the circumference of the outer stent.

18. The heart valve prosthesis of claim 2, wherein the outer stent further comprises a third row of third outer nodes, wherein the third row of third outer nodes is disposed between the first row of first angled outer struts and the second row of second angled outer struts.

19. The heart valve prosthesis of claim 18, wherein the outer stent includes a first row of first outer cells, wherein each first cell of the first row of outer cells is defined by a single one of the third outer nodes, exactly two of the second angled outer struts, exactly two of the first outer nodes, exactly two of the third angled outer struts, and a single one of the second outer nodes.

20. The heart valve prosthesis of claim 19, wherein the first row of first outer cells consists of exactly twenty-four first outer cells.

21. The heart valve prosthesis of claim 18, wherein the outer stent further includes fourth row of fourth angled outer struts, wherein the second outer nodes are disposed between the third angled outer struts and the fourth angled outer struts.

22. The heart valve prosthesis of claim 21, wherein each of the second outer nodes is coupled to a first end of a single fourth angled outer strut of the fourth row of fourth angled outer struts.

23. The heart valve prosthesis of claim 22, wherein the outer stent further includes a fourth row of fourth outer nodes, wherein each fourth outer node is coupled of a second end of a pair of the fourth angled outer struts.

24. The heart valve prosthesis of claim 23, wherein the outer stent further include a second row of second outer cells, wherein each second outer cell is defined by a single one first outer nodes, two third angled outer struts, two second outer nodes, two fourth angled outer struts, and a single one of the fourth outer nodes.

25. The heart valve prosthesis of claim 24, wherein the second row of second outer cells consists of exactly twelve second outer cells.
